(19) United States
(12) Patent Application Publication
(10) Pub. No.: US 2026/0102440 A1
(43) Pub. Date: Apr. 16, 2026

(54) PROBIOTIC STRAINS FOR THE TREATMENT OF IRRITABLE BOWEL SYNDROME AND FODMAP INTOLERANCE

(71) Applicant: Evonik Operations GmbH, Essen (DE)

(72) Inventors: Bodo Speckmann, Kahl (DE); Marco Gobbetti, Bozen-Bolzano (IT); Heike Tom Dieck, Friedrichsdorf (DE); Iris Wiegräbe, Bielefeld (DE)

(73) Assignee: Evonik Operations GmbH, Essen (DE)

(21) Appl. No.: 19/115,010

(22) PCT Filed: Sep. 15, 2023
(86) PCT No.: PCT/EP2023/075382

§ 371 Date: Mar. 25, 2025

(30) Foreign Application Priority Data
Sep. 30, 2022 (EP) 22198956.9

(51) Int. Cl. A61K 35/747; A23L 29/00; A61K 35/00; A61K 35/742; A61K 38/48; A61P 1/00

(52) U.S. Cl. CPC A61K 35/747; A23L 29/065; A61K 35/742; A61K 38/48; A61K 38/482; A61P 1/00; A23V 2400/165; A23V 2400/169; A23V 2400/173; A61K 2035/115

(57) ABSTRACT

The present invention relates to methods of treatment or prevention of irritable bowel syndrome (IBS), abdominal pain, disordered defecation, and FODMAP intolerance, by administering a preparation containing probiotic strain of Lactobacillus plantarum (Lactiplantibacillus plantarum) DSM 33363, or the preparation in combination with Lactobacillus plantarum (Lactiplantibacillus plantarum) DSM 33364, Lactobacillus paracasei (Lacticaseibacillus paracasei) DSM 33373, Lactobacillus resuteri (Limosilactobacillus reuteri) DSM 33374, Bacillus megaterium (Priestia megaterium) DSM 33300, Bacillus pumilus DSM 33297, and Bacillus pumilus DSM 33355, as viable cells or cytoplasmic extract thereof. A foodstuff composition containing the preparation disclosed herein is also described.

This invention concerns preparations comprising the probiotic strains Lactobacillus plantarum (Lactiplantibacillus plantarum) DSM 33363, Lactobacillus plantarum (Lactiplantibacillus plantarum) DSM 33364, Lactobacillus paracasei(Lacticaseibacillus paracasei) DSM 33373, Lactobacillus reuteri (Limosilactobacillus reuteri) DSM 33374, Bacillus megaterium (Priestia megaterium) DSM 33300, Bacillus pumilus DSM 33297, Bacillus pumilus DSM 33355 as viable cells or cytoplasmic extract thereof and their use for treating and preventing irritable bowel syndrome (IBS) and intolerance towards fermentable oligosaccharides, disaccharides, monosaccharides, and polyols (FODMAP) in humans and animals.

IBS is a highly prevalent functional gastrointestinal disorder, also called a disorder of the gut-brain interaction, affecting up to 9% of the general population in the US, Canada, and Great Britain¹, and which is characterized by recurrent abdominal pain and disordered defecation. IBS significantly impairs the quality of life of affected individuals and poses a huge burden to health care systems worldwide, thus highlighting the need for novel effective measures to treat and prevent this disease. According to an American College of Gastroenterology clinical guideline for the management of IBS, the therapeutic consensus strategy comprises a low FODMAP diet, treatment of constipation and diarrhea symptoms by the use of chloride channel activators/guanylate cyclase activators and rifaximin, respectively, as well as the use of gut-directed psychotherapy to treat global IBS symptoms². Of note, this clinical guideline demonstrates that treatments are only symptomatic and that at present no cure exists for IBS. IBS is particularly difficult to treat due to its multifactorial nature, including gut dysbiosis, imbalanced neurotransmitter levels and functions affecting intestinal motility, visceral sensitivity, and gut-brain interaction, and an impairment of the intestinal barrier integrity.

Nutritional treatment strategies are largely exclusion diets such as FODMAP-free, gluten-free, and dairy-free diets³, which are difficult to adhere to, are questionable in their efficacy⁴ and only partially address the etiology of this disease. Also, probiotics of the genera Lactobacillus, Bifidobacterium, and Bacillus coagulans have been applied with mixed success, as assessed in a meta-analysis of randomized controlled trials⁵. The primary rationale for using probiotics in this context has been to target IBS-associated dysbiosis^{6,7}, as is the subject of several publications as follows.

Recently, the taxonomic classification of several species of the genera Lactobacillus and Bacillus has been updated⁸⁻¹⁰. Of relevance in the context of this invention are the following species:

| “Old” denomination | Updated denomination (since 2020) |
|---|---|
| Lactobacillus paracasei | Lacticaseibacillus paracasei |
| Lactobacillus plantarum | Lactiplantibacillus plantarum |
| Lactobacillus reuteri | Limosilactobacillus reuteri |
| Bacillus megaterium | Priestia megaterium |
| Bacillus pumilus | Bacillus pumilus |

For convenience, in the example part the old denomination will be used, whereas both denominations will be used in the general description and in the claims section.

U.S. Pat. No. 12,001,603A1 describes preparations comprising fibers and polyethylene glycol in combination with various generic probiotic strains of the genera Lactobacillus, Bifidobacterium, and Bacillus as alleged treatments for IBS but fails to disclose if or how the mentioned probiotics themselves would have an influence on the disease. WO2017212433 claims compositions comprising bacteria of the genera Lactobacillus and Bifidobacterium, including the species Lactobacillus plantarum (Lactiplantibacillus plantarum), Lactobacillus paracasei (Lacticaseibacillus paracasei), and Lactobacillus reuteri (Limosilactobacillus reuteri) for treating IBS. Xie et al. assessed low FODMAP diet and probiotics as possible treatments for the relief of IBS symptoms⁵, among the group of probiotics they referred to e.g. the genera Bacillus (coagulans) and Lactobacillus individually but not to a possible combination of both. Another evaluation of existing clinical trials conducted with IBS patients proposes combinations of Lactobacillus rhamnosus and Lactobacillus acidophilus as particularly promising⁷. Compositions of Bacillus megaterium (Priestia megaterium) and polyunsaturated fatty acid salts and their use as e.g., IBS treatments have been disclosed by Speckmann et al.¹¹ and WO/2020/109474. A Lactobacillus reuteri (Limosilactobacillus reuteri) ATCC 55730 strain has been assessed in a trial with IBS patients¹². Similarly, a combination of Lactobacillus paracasei(Lacticaseibacillus paracasei) HA-196 together with a Bifidobacterium R0175 strain has been assessed in IBS patients¹³. To the best of our knowledge, strains of the species Bacillus megaterium (Priestia megaterium) and Bacillus pumilus, alone or in combination with any other (Bacillus or non-Bacillus) probiotic strain(s) for use in the management and/or prevention of IBS has not been disclosed so far.

Numerous other patent applications disclose synbiotic compositions, typically defined as combinations of probiotics and prebiotics, as possible treatments of IBS and/or other chronic diseases of the gastrointestinal tract. For example, EP10182284B1 discloses a composition of fermented cereal grains together with Lactobacillus plantarum (Lactiplantibacillus plantarum) 299 for the treatment of IBS. WO2011078781 discloses synbiotic compositions comprising a cereal-derived prebiotic and Lactobacillus strains for use in treatment of e.g. IBS.

The limitation of the strategy mentioned above is that the cause-and-effect relationship of gut dysbiosis and IBS is somewhat unclear, gut dysbiosis, or rather the composition of the gut microbiota, is affected by numerous other dietary and intrinsic (e.g., genetic) factors, and the impact of probiotics in this context is dependent on their survivability and the composition of the resident microbiota.

The goal of the present invention was to provide a probiotic composition which survives under conditions of the stomach and small intestine for the prevention and treatment of IBS and symptoms related to IBS.

Our invention is based on a more rationale selection, development, and application of probiotic strains that target multiple features of IBS beyond e.g., dysbiosis. We conceived that an efficacious treatment strategy needs to target multiple of the pathophysiological features of IBS, must be easily accessible and integrable into the patient's lifestyle.

Under WO/2021/129998 and ¹⁴ we disclosed previously a combination of Lactobacillus plantarum (Lactiplantibacillus plantarum) DSM 33363, Lactobacillus plantarum (Lactiplantibacillus plantarum) DSM 33364, Lactobacillus paracasei (Lacticaseibacillus paracasei) DSM 33373, Lactobacillus reuteri (Limosilactobacillus reuteri) DSM 33374, Bacillus megaterium (Priestia megaterium) DSM 33300, Bacillus pumilus DSM 33297, Bacillus pumilus DSM 33355 (=the IBS consortium), among other combinations³, and its capability to fully digest gluten and its use for treating the so-called gluten-dependent IBS. However, it is to be no noted that although the clinical picture may be similar, the mechanism underlying the so-called gluten-dependent IBS differs significantly from “real” IBS: The symptoms of gluten-dependent IBS are triggered by peptides derived from partial gluten digestion with toxic and/or immunogenic activity. In fact, the majority of IBS patients do not respond to a gluten-free diet, whereas a gluten-dependency is indicative of either celiac disease, non-celiac gluten/wheat sensitivity, or wheat allergy¹⁵. Accordingly, gluten-dependency is an important differentiator in the diagnosis, etiology and treatment of IBS.

Unexpectedly, we found out that the IBS consortium has functionalities that makes it an effective treatment for IBS, i.e. for subtypes not related to the ingestion and/or gastrointestinal metabolization of gluten. These functionalities include the production of the neurotransmitter γ-aminobutyric acid (GABA) from various food matrices (whole bread, white bread, wheat flour) upon simulated gastrointestinal digestion. GABA is a crucial regulator of gut motility, has visceral anti-nociceptive functions^{16,17}, and its levels are diminished in IBS patients¹⁸. The production of GABA by the IBS consortium was severalfold stronger than compared to control conditions and to other probiotic consortia.

Moreover, we found that strains of the IBS consortium can grow on various types of FODMAP, the fructans inulin and fructooligosaccharides (FOS) in particular, thus degrading them before they can reach the large bowel and cause the typical symptoms of IBS.

Lastly, the IBS consortium beneficially modulates the function and survival of the intestinal barrier, as determined in vitro. In a model of the gastrointestinal tract, we observed that digests of wheat-based foodstuffs (white bread, whole bread, wheat flour) impair intestinal barrier integrity, which was not rescued by addition of digestive proteases or an (alternative) microbial consortium, whereas the IBS consortium completely rescued the adverse effects of (partially) digested foodstuffs.

Overall, we here discovered that a consortium of probiotic strains previously disclosed by us has unique and surprising functions encompassing the release of GABA, digestion of FODMAP, and protection of the gastrointestinal barrier integrity. Our probiotic strains display very good survivability in simulated stomach and small intestinal conditions, and a very good storage stability, which altogether makes this consortium a promising novel ingredient for preparations (dietary supplements, functional foods as well as drugs) to treat and or prevent IBS and FODMAP intolerance.

Therefore, the present invention is directed to a preparation comprising Lactobacillus plantarum (Lactiplantibacillus plantarum) DSM 33363.for use in a method of treatment and/or prevention of irritable bowel syndrome (IBS).

In accordance with the above, the present invention also provides a method of treatment and/or prevention of irritable bowel syndrome (IBS), the method comprising administering preparation comprising Lactobacillus plantarum (Lactiplantibacillus plantarum) DSM 33363.

IBS is preferably independent from the ingestion and/or gastrointestinal metabolization of gluten and/or physiological response to gluten or fragmented gluten.

In one embodiment of the present invention, the preparation comprises further one or more of the strains Lactobacillus plantarum (Lactiplantibacillus plantarum) DSM 33364, Lactobacillus paracasei (Lacticaseibacillus paracasei) DSM 33373, Lactobacillus reuteri (Limosilactobacillus reuteri) DSM 33374, Bacillus megaterium (Priestia megaterium) DSM 33300, Bacillus pumilus DSM 33297, and Bacillus pumilus DSM 33355.

In a preferred embodiment, the preparation comprises the IBS consortium, including Lactobacillus plantarum DSM 33363, Lactobacillus plantarum (Lactiplantibacillus plantarum) DSM 33364, Lactobacillus paracasei (Lacticaseibacillus paracasei) DSM 33373, Lactobacillus reuteri DSM 33374, Bacillus megaterium (Priestia megaterium) DSM 33300, Bacillus pumilus DSM 33297, and Bacillus pumilus DSM 33355.

The therapeutic effect of these preparations further includes the improvement of IBS symptoms, such as recurrent abdominal pain, disordered defecation, constipation, diarrhea, flatulence, and bloating. The therapeutic effect also encompasses the amelioration of intolerance towards FODMAP.

The therapeutic effects of said preparations are achieved via one or more of the following modes of action:
a) the preparations induce the formation of GABA from various food components within the gastrointestinal tract. The source of the food components can be foodstuffs based on wheat as well as on other cereal grains, for example.
b) The preparations digest FODMAP such as inulin and FOS in the upper parts of the gastrointestinal tract, thereby preventing them from reaching the large intestine and causing the symptoms of IBS
c) The preparations improve the barrier function of the intestinal surface.

In one embodiment, the preparation according to the present invention is for use in the treatment or prevention of symptoms associated with IBS, preferably abdominal pain, and disordered defecation.

In one embodiment, the preparation according to the present invention is for use in the treatment or prevention of intolerance towards fermentable oligosaccharides, disaccharides, monosaccharides, and polyols (FODMAP intolerance)

The cells of the strains of the current invention may be present in the compositions of the current invention, as spores (which are dormant), as vegetative cells (which are growing), as transition state cells (which are transitioning from vegetative cells to spores, or reverse), as cellular extracts or as a combination of at least two of these types of cells. In a preferred embodiment, the probiotic strain is present in a dormant form or as vegetative cells. In alternative embodiment, cytoplasmic extracts or cell-free supernatants or heat-killed biomass of the probiotic strains are used.

In an alternative embodiment, the preparations further comprise one or more probiotic strains.

In a further preferred embodiment, the preparations further comprise one or more of the following: microbial proteases purified from Aspergillus niger, Aspergillus oryzae, Bacillus sp., Lactobacillus sp., Pediococcus sp., Weissella sp., Rothia mucilaginosa, Rothia aeria, subtilisins, nattokinase.

In an alternative embodiment, the preparation further comprises enzymes that facilitate the digestion of carbohydrates, proteins, peptides, lipids.

In a preferred embodiment, the preparation for use further comprises a substance, which acts as permeabilizer of the microbial cell membrane of members of Bacillus sp., Lactobacillus sp., Pediococcus sp., Weissella sp., preferably alginate.

In an alternative embodiment, one or more of the probiotic strains selected from Bacillus sp. and Lactobacillus sp. are immobilized individually or as consortia. Immobilization can be realized on solid surfaces such as cellulose and chitosan, as entrapment within a porous matrix such as polysaccharide gels like alginates, k-carrageenan, agar, chitosan and polygalacturonic acid or other polymeric matrixes like gelatin, collagen and polyvinyl alcohol or by flocculation and microencapsulation or electrospraying technologies.

In another preferred embodiment, the preparation is for treating or preventing intolerance towards fermentable oligosaccharides, disaccharides, monosaccharides, and polyols (FODMAP), including fructose intolerance, in a subject in need thereof.

In another embodiment, one or more of the probiotic strains selected from Bacillus sp. or Lactobacillus sp. are immobilized individually or as consortia.

One subject of the present invention is the use of a preparation according to the present invention as a food or feed supplement or functional food or food product or pharmaceutical product. Preferred foodstuffs according to the invention are cereals, bread, chocolate products, gummies, mueslis, muesli bars, health bars, biscuits, spreads, and dairy products.

Therefore, in a preferred embodiment, the preparation is formulated for oral use, preferably as pills, capsules, tablets, granular powders, opercula, soluble granules, bags, pills or drinkable vials, or is formulated as syrup or beverage, or is added to food, preferably cereals, gummies, bread, muesli, muesli bars, health bars, biscuits, chocolates, yoghurts or spreads.

A further subject of the current invention is also the use of a preparation of the current invention as a synbiotic ingredient in food products.

A further subject of the present invention is a foodstuff composition containing a preparation according to the present invention and at least one further food ingredient, preferably selected from proteins, carbohydrates, fats, further probiotics, prebiotics, enzymes, vitamins, immune modulators, milk replacers, minerals, amino acids, coccidiostats, acid-based products, medicines, and combinations thereof.

The foodstuff composition according to the present invention does also include dietary supplements, e. g. in the form of a pill, capsule, tablet, powder, sachet, opercula, soluble granules, bags, or drinkable vials, syrup, beverage, or other liquids.

In another preferred embodiment, the preparation is used to reduce the FODMAP content of food products derived from cereal grains wheat, oat, rye, barley during the production of these foods.

### WORKING EXAMPLES

### Example 1: Probiotic Strains Resistant to Gastrointestinal Conditions

Probiotic strains were exposed to conditions simulating the stomach and small intestine as detailed by De Angelis et al.¹⁴ with an initial cell density of 10⁹ CFU/ml. The seven strains listed in Table 1 showed very good survival under both tested conditions and stood out against the majority of the in total 504 strains tested in¹⁴. We thereby verified that the probiotic strains that are part of this invention can strive under conditions of the stomach and small intestine to ensure their sustained efficacy in humans.

TABLE 1
Survival of probiotic strains during simulated gastric and small intestinal conditions

| | | Gastric digestion | | | | Small intestinal digestion | | | |
|---|---|---|---|---|---|---|---|---|---|
| | | (CFU log₁₀) | | | | (CFU log₁₀) | | | |
| | | | | pH | | | | pH | |
| Species | Strain | pH 2 | pH 3 | 2SM | pH 8 | pH 2 | pH 3 | 2SM | pH 8 |
| Bacillus megaterium | DSM33300 | 8 | 8.9 | 8.1 | 8.5 | 8 | 8.9 | 8.1 | 8.5 |
| Bacillus pumilus | DSM33297 | 6.69 | 8.18 | 8.76 | 8.76 | 6.6 | 7.11 | 7.91 | 7.85 |
| Bacillus pumilus | DSM33335 | 7.89 | 8.11 | 8.32 | 8.32 | 7.4 | 8.02 | 8 | 8.26 |
| Lactobacillus | DSM33373 | 8.12 | 8.54 | 9.28 | 9.52 | 7.35 | 7.74 | 9.31 | 9.31 |
| paracasei | | | | | | | | | |
| Lactobacillus | DSM33364 | 7.2 | 8.3 | 8.8 | 9 | 6.9 | 8.5 | 8.2 | 9.3 |
| plantarum | | | | | | | | | |
| Lactobacillus | DSM33363 | 7.2 | 8.1 | 8.9 | 9.2 | 6.89 | 8.29 | 7.8 | 9.2 |
| plantarum | | | | | | | | | |
| Lactobacillus reuteri | DSM33374 | 7.22 | 8.56 | 9.04 | 9.13 | 7 | 8.34 | 8.4 | 8.2 |

### Example 2: Production of GABA from Food by Probiotic Strains During Simulated Gastrointestinal Conditions

### Digestion of Foodstuffs (Wheat Flour, White Bread, Whole Bread) Under Simulated Gastrointestinal Conditions and Quantitative Assessment of GABA

Three foodstuffs (Gluten prepared from wheat flour, white and whole wheat bread (chewed for 30 s and collected in a beaker with 10 ml of NaK-phosphate 0.05 M, pH 6.9)) were subjected to simulated gastrointestinal conditions as described by De Angelis et al.¹⁴ with or without addition of probiotic strains. Digested samples of gluten, white and whole wheat breads were assayed for the content of GABA contained in the pH 4.6-soluble nitrogen fraction by a Biochrom 30 series amino acid analyzer (Biochrom Ltd., Cambridge Science Park, England) with a sodium cation-exchange column (20 by 0.46 cm [inner diameter]). A concentration range of GABA (Sigma Chemical Co., Milan, Italy) was used as standard. Proteins and peptides in the samples were precipitated by addition of 5% (vol/vol) cold solid sulfosalicylic acid, holding the samples at 4° C. for 1 h, and centrifuging them at 15,000×g for 15 min. The supernatant was filtered through a 0.22-μm-pore-size filter and diluted, when necessary, with sodium citrate (0.2 M, pH 2.2) loading buffer. Amino acids were post-column derivatized with ninhydrin reagent and detected by absorbance at 440 (proline and hydroxyproline) or 570 (all the other amino acids and GABA) nm. The capacity for synthesizing GABA by strains was further assayed in MRS with and without the supplementation with glutamate. This ability was also tested after gastrointestinal digestion of gluten, white and whole wheat bread.

### Results

All the strains tested produced considerable concentrations of GABA, with Lactobacillus plantarum (Lactiplantibacillus plantarum) DSM 33363 producing by far the highest level, with on average ˜sixfold more than all other strains. For this reason, the strain was then tested in further conditions to determine how different substrates-gluten prepared from wheat flour, whole wheat bread, MRS medium±glutamate- are used by the strain for production of GABA. As shown in Panel B, L. plantarum DSM33363 was able to produce GABA from all substrates.

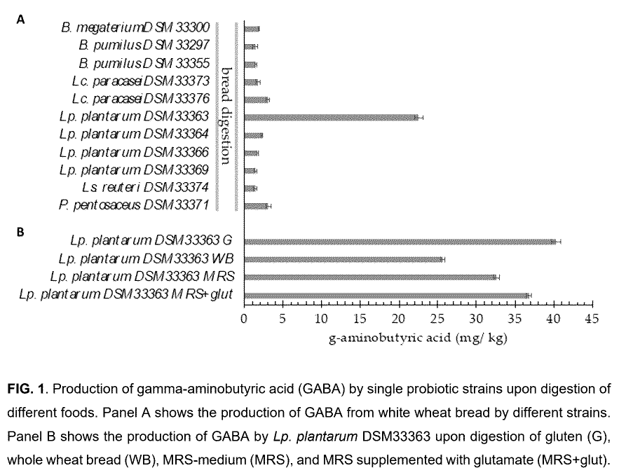

FIG. 1 shows the production of gamma-aminobutyric acid (GABA) by single probiotic strains upon digestion of different foods. Panel A shows the production of GABA from white wheat bread by different strains. Panel B shows the production of GABA by Lp. plantarum DSM33363 upon digestion of gluten (G), whole wheat bread (WB), MRS-medium (MRS), and MRS supplemented with glutamate (MRS+glut).

Likewise, the GABA-producing capability of the Lp. plantarum DSM33363-comprising IBS consortium is much higher as compared to another consortium (comprising Lp. plantarum DSM33366, DSM33369; Ls. reuteri DSM33374; Lc. paracasei DSM33376; Pediococcus (P.) pentosaceus DSM33371; B. pumilus DSM33297, DSM33355) and also compared to two different protease preparations. This difference was irrespective of the substrate and therefore an intrinsic feature of the IBS consortium.

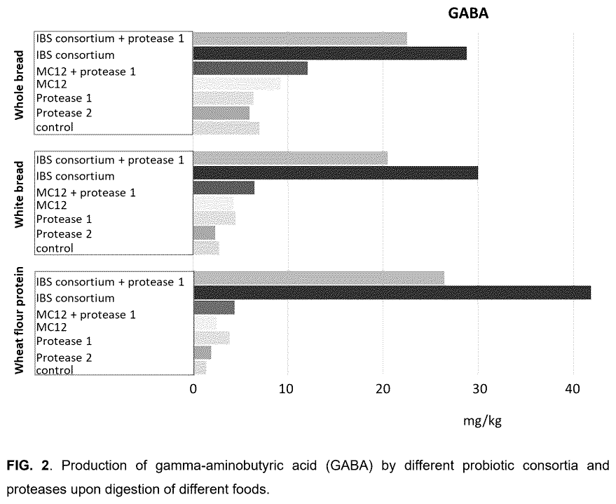

FIG. 2 shows the production of gamma-aminobutyric acid (GABA) by different probiotic consortia and proteases upon digestion of different foods.

### Example 3: Fecal Levels of GABA Upon Supplementation with Probiotic Strains Versus Placebo

### Human Trial Assessing the Impact of Gluten Challenge Trial Outline

Healthy human individuals aged 18-50 years received either probiotic (N=50) or placebo (N=20) capsules, dosed as one capsule per day for in total 34 days followed by a period of 7 days of wash-out. Probiotic capsules contained a formulation of the IBS consortium with at least 3×10⁹ CFU in total, which was ensured over the whole trial period by regular testings. Faecal samples were collected at various time points for quantification of GABA, performed as detailed above.

### Results

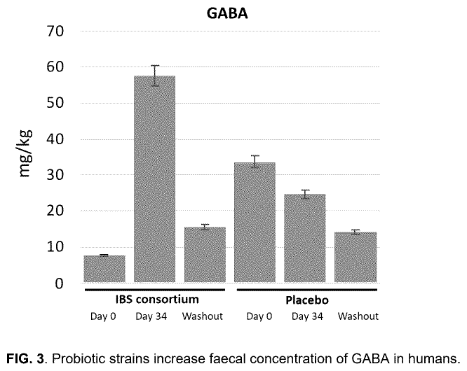

FIG. 3 shows the probiotic strains increase faecal concentration of GABA in humans.

Concentration of GABA found in faecal samples delivered by 8 volunteers (4 treated with placebo (PL) and 4 treated with probiotics (IBS consortium)) at baseline (day 0), after 34 days of daily ingestion of one capsule filled with probiotic strains (day 34) and following a washout period of seven days (washout). The fecal concentration of GABA in the placebo arm markedly reduced during the trial. Oppositely, in the verum arm, the concentration of GABA increased ˜sevenfold after 34 days of probiotic treatment. Seven days after ablation the GABA concentration decreased to near baseline levels.

### Example 4: Ability of Probiotic Strains to Use FODMAP as Growth Substrates

Growth of Probiotic Strains on Minimal Growth Medium Supplemented with FODMAP

Low glucose media (LBG with 0.1 g/L glucose for Bacillus sp., MRS with 2 g/L glucose for Lactobacillus sp.) were used as minimal growth media and supplemented with or without 5 g/L of FODMAP (β-glucans, FOS, or inulin) in comparison to control media with high glucose content of 20 g/L. Strains were cultured for 24 hours, CFU concentrations were determined by the plate count methodology using serial dilutions.

### Quantification of SCFA

One mL of supernatant of culture strains (obtained after centrifugation of culture strains at 10.000 rpm for 10 min), added with 10 μL of internal standard solution (4-methyl-2-pentanol) at 33 ppm, was placed into 20 mL glass vials and sealed with polytetrafluoroethylene (PTFE)-coated silicone rubber septa (20 mm diameter) (Supelco, Bellefonte, PA, USA). A micro-extraction procedure was performed and the extracted compounds were desorbed in splitless mode for 3 minutes at 220° C. A Clarus 680 (Perkin Elmer) gas-chromatography was equipped with a capillary column Rtx-WAX (30 m×0.25 mm i.d., 0.25 μm film thickness) (Restek, Bellfonte, PA, USA). The column temperature was set initially at 35° C. for 8 min, then increased to 60° C. at 4° C. min−1, to 160° C. at 6° C. min−1, and finally to 200° C. at 20° C. min−1, and held for 15 min. Helium was used as the carrier gas at flow rate of 1 mL min−1. The single quadrupole mass spectrometer Clarus SQ 8C (Perkin Elmer) was coupled to the gas chromatography system. The source and transfer line temperatures were kept at 250 and 230° C., respectively. Electron ionization masses were recorded at 70 eV in the m/z (mass-to-charge ratio) interval from 34 to 350. The GC-MS generated a chromatogram with peaks representing individual compounds. Each chromatogram was analyzed for peak identification using the National Institute of Standard and Technology 2008 (NIST) library. A peak area threshold >1 000 000 and 90% or greater probability of match was used for VOC identification followed by manual visual inspection of the fragment patterns. Quantitative data for the compounds identified were obtained by the interpolation of the relative areas versus the internal standard area.

### Results

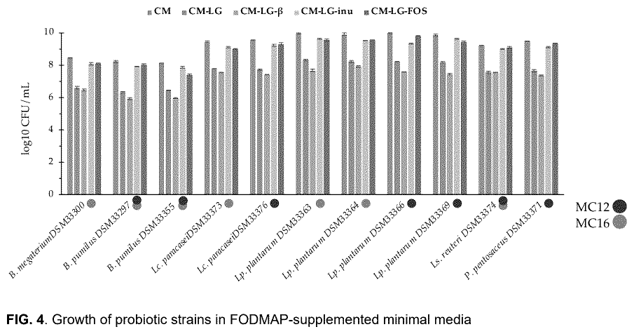

FIG. 4 shows the growth of probiotic strains in FODMAP-supplemented minimal media.

All tested strains grew in low-glucose medium (CM-LG) supplemented with inulin (inu) or FOS to similar levels as in the high glucose control medium (CM), while β-glucan supplementation (CM-LG-β) in general had no growth-stimulatory effect. As shown in Figure__, the strains DSM 33363 and DSM 33374 were exceptionally efficient in metabolizing inulin and FOS, respectively, as revealed by increased formation of acetate.

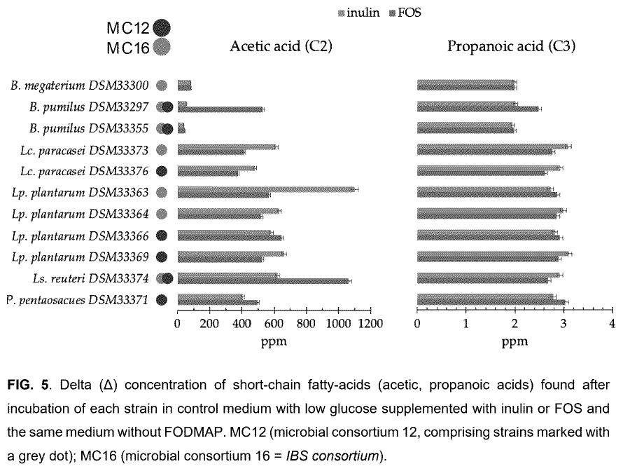

FIG. 5 shows the Delta (Δ) concentration of short-chain fatty-acids (acetic, propanoic acids) found after incubation of each strain in control medium with low glucose supplemented with inulin or FOS and the same medium without FODMAP. MC12 (microbial consortium 12, comprising strains marked with a grey dot); MC16 (microbial consortium 16=IBS consortium).

### Example 5: Impact of Food Digested Under Simulated Gastrointestinal Conditions on Intestinal Barrier Integrity and its Modulation by Probiotic Strains

### Preparation of Caco-2 Cells for Membrane Integrity Assay on Trans Well Plates

Caco-2 cell line was obtained from the German Collection of Microorganisms and Cell Cultures GmbH (DSMZ, Braunschweig, Germany) ACC 169. Cells were cultured in MEM (Sigma Aldrich #51416C) supplemented with 2 mM L-glutamine, 10% (v/v) FBS, 1% (v/v) non-essential amino acids, 1% (v/v) penicillin/streptomycin.Caco-2 cells were grown in 25-cm² and/or 75-cm² T-flasks and maintained in culture at 37° C. in a humidified 5% CO2-95% air atmosphere. They were subcultured at 80-90% confluence every 3-4 days. Near confluence, cells were detached with trypsin, counted, and seeded at a density of 1×10⁵ cells per 500 μl onto 12 mm polycarbonate membrane Transwell inserts with 0.4 μm pore size (Corning®). Cells were cultured for 21 days to reach differentiation, and growth media was refreshed every 2-3 days. TEER value was monitored by a Millicell® ERS meter (Merck KGaA, Darmstadt, Germany). The monolayer was considered to be confluent when the value reached a plateau with a reading ˜600-800 Ohm/cm². The confluent monolayers were then used for further experiments.

Digest samples of foodstuff (gluten/wheat flour and white wheat bread) with two different probiotic consortia (IBS consortium and MC12) were investigated regarding their impact on barrier integrity in a Caco-2 cells Monolayer Assay as described above. Triplicates were prepared of all approaches.

### Results

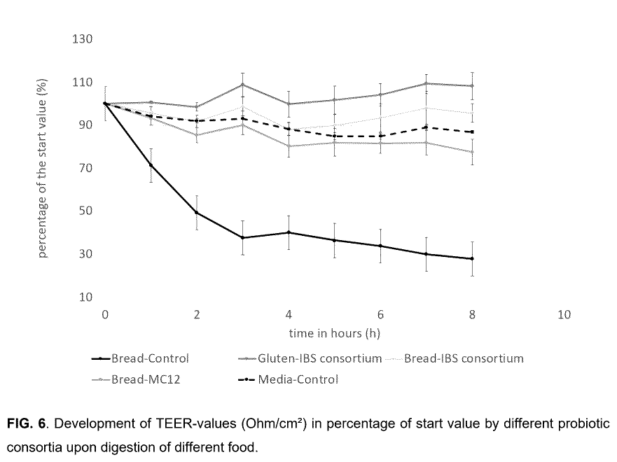

FIG. 6 shows the development of TEER-values (Ohm/cm²) in percentage of start value by different probiotic consortia upon digestion of different food.

The Bread-Control without probiotic consortia containing residual gluten showed total damage of the barrier integrity resulting in decreasing TEER values in the first three hours. Whereas the different probiotic consortia in different food (gluten, white bread), which contained no residual gluten, showed stable barrier integrities in comparison with the untreated media control. The IBS consortium was even better as the media control (untreated cells) and the probiotic consortium MC12.

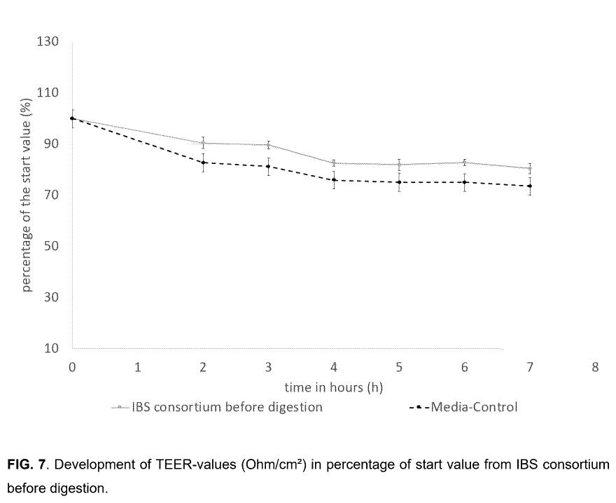

FIG. 7 shows the development of TEER-values (Ohm/cm²) in percentage of start value from IBS consortium before digestion.

The IBS consortium before digestion stabilized the barrier integrity of the monolayer and has therefore on its own a positive impact.

### Example 6: Unique Amino Acid Profile of Dough Digested by the IBS Consortium Versus MC12- or Enzyme-Digested Dough

### Preparation of Dough Digested by Enzymes or Microbial Consortia and Subsequent Amino Acid Profiling

10 g of gluten-enriched powdered extract from wheat flour was added to saliva pooled from three subjects and placed into beakers containing 10 mL of NaK buffer solution (0.05 M, pH 6.9), followed by mechanical homogenization with a lab stomacher for 30 sec. Microbial consortia were added to each suspension. Additionally, control samples containing the afore-mentioned commercial enzymes (Promod™ or Tolerase® G) without bacterial cells were also included. The saliva-containing doughs were added to simulated gastric juice, which included NaCl (125 mM), KCl (7 mM), NaHCO3 (45 mM), and pepsin (3 g/L), while pH was adjusted at 2 by using 0.1 M HCl. Samples were incubated at 37° C. under stirring conditions (200×g) simulating peristalsis. After 180 min, a simulated intestinal juice (pH 8.0) containing 0.1% (w/v) pancreatin (Sigma-Aldrich Co.) and 0.15% (w/v) Oxgall bile salts (Sigma-Aldrich Co.) was added to each sample and maintained at 37° C. under stirring conditions (200×g). These conditions, simulating the intestinal phase, was prolonged till 48 h (that is, 3 h of gastric phase and 45 h of intestinal phase).

Amino acid quantification was performed as described in Example 2, whereby amino acids were post-column derivatized with ninhydrin reagent and detected by absorbance at 440 nm (proline and hydroxyproline) or 570 nm (all the other amino acids).

### Results

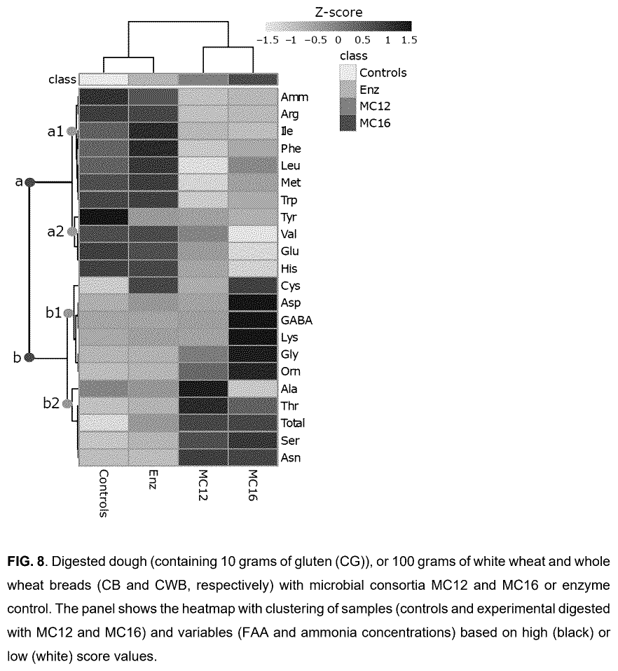

FIG. 8 shows that the application of the IBS consortium (=MC16) lead to high levels of Asp, GABA, Lys, Gly and Orn, whereas it strongly depleted the amino acids L-histidine and L-glutamic acid, biomarkers and exacerbators of IBS,^{19,20} respectively, during simulated gluten digestion, and that these surprising effect were not seen with a related consortium (MC12) or under control conditions without added bacterial cells.

Digested dough (containing 10 grams of gluten (CG)), or 100 grams of white wheat and whole wheat breads (CB and CWB, respectively) with microbial consortia MC12 and MC16 or enzyme control. The panel shows the heatmap with clustering of samples (controls and experimental digested with MC12 and MC16) and variables (FAA and ammonia concentrations) based on high (black) or low (white) score values.

### LITERATURE

1. Palsson, O. S., Whitehead, W., Tornblom, H., Sperber, A. D. & Simren, M. Prevalence of Rome IV Functional Bowel Disorders Among Adults in the United States, Canada, and the United Kingdom. Gastroenterology 158, 1262-1273 e1263 (2020).
2. Lacy, B. E., et al. ACG Clinical Guideline: Management of Irritable Bowel Syndrome. Am J Gastroenterol 116, 17-44 (2021).
3. Lenhart, A., et al. Effect of Exclusion Diets on Symptom Severity and the Gut Microbiota in Patients With Irritable Bowel Syndrome. Clin Gastroenterol Hepatol 20, e465-e483 (2022).
4. Dionne, J., et al. A Systematic Review and Meta-Analysis Evaluating the Efficacy of a Gluten-Free Diet and a Low FODMAPs Diet in Treating Symptoms of Irritable Bowel Syndrome. Am J Gastroenterol 113, 1290-1300 (2018).
5. Xie, C. R., et al. Low FODMAP Diet and Probiotics in Irritable Bowel Syndrome: A Systematic Review With Network Meta-analysis. Front Pharmacol 13, 853011 (2022).
6. Principi, N., Cozzali, R., Farinelli, E., Brusaferro, A. & Esposito, S. Gut dysbiosis and irritable bowel syndrome: The potential role of probiotics. J Infect 76, 111-120 (2018).
7. Ceccherini, C., Daniotti, S., Bearzi, C. & Re, I. Evaluating the Efficacy of Probiotics in IBS Treatment Using a Systematic Review of Clinical Trials and Multi-Criteria Decision Analysis. Nutrients 14(2022).
8. Zheng, J., et al. A taxonomic note on the genus Lactobacillus: Description of 23 novel genera, emended description of the genus Lactobacillus Beijerinck 1901, and union of Lactobacillaceae and Leuconostocaceae. Int J Syst Evol Microbiol 70, 2782-2858 (2020).
9. Patel, S. & Gupta, R. S. A phylogenomic and comparative genomic framework for resolving the polyphyly of the genus Bacillus: Proposal for six new genera of Bacillus species, Peribacillus gen. nov., Cytobacillus gen. nov., Mesobacillus gen. nov., Neobacillus gen. nov., Metabacillus gen. nov. and Alkalihalobacillus gen. nov. Int J Syst Evol Microbiol 70, 406-438 (2020).
10. Gupta, R. S., Patel, S., Saini, N. & Chen, S. Robust demarcation of 17 distinct Bacillus species clades, proposed as novel Bacillaceae genera, by phylogenomics and comparative genomic analyses: description of Robertmurraya kyonggiensis sp. nov. and proposal for an emended genus Bacillus limiting it only to the members of the Subtilis and Cereus clades of species. Int J Syst Evol Microbiol 70, 5753-5798 (2020).
11. Speckmann, B., et al. Synbiotic Compositions of Bacillus megaterium and Polyunsaturated Fatty Acid Salt Enable Self-Sufficient Production of Specialized Pro-Resolving Mediators. Nutrients 14(2022).
12. Niv, E., Naftali, T., Hallak, R. & Vaisman, N. The efficacy of Lactobacillus reuteri ATCC 55730 in the treatment of patients with irritable bowel syndrome—a double blind, placebo-controlled, randomized study. Clin Nutr 24, 925-931 (2005).
13. Lewis, E. D., et al. Efficacy of Lactobacillus paracasei HA-196 and Bifidobacterium longum R0175 in Alleviating Symptoms of Irritable Bowel Syndrome (IBS): A Randomized, Placebo-Controlled Study. Nutrients 12(2020).
14. De Angelis, M., et al. Selection of Gut-Resistant Bacteria and Construction of Microbial Consortia for Improving Gluten Digestion under Simulated Gastrointestinal Conditions. Nutrients 13(2021).
15. Czaja-Bulsa, G. Non coeliac gluten sensitivity—A new disease with gluten intolerance. Clin Nutr 34, 189-194 (2015).
16. Laroute, V., et al. Lactococcus lactis NCDO2118 exerts visceral antinociceptive properties in rat via GABA production in the gastro-intestinal tract. Elife 11(2022).
17. Chen, M., et al. Neurotransmitter and Intestinal Interactions: Focus on the Microbiota-Gut-Brain Axis in Irritable Bowel Syndrome. Front Endocrinol (Lausanne) 13, 817100 (2022).
18. Aggarwal, S., Ahuja, V. & Paul, J. Dysregulation of GABAergic Signalling Contributes in the Pathogenesis of Diarrhea-predominant Irritable Bowel Syndrome. J Neurogastroenterol Motil 24, 422-430 (2018).
19. Yu, Q., et al. Mass spectrometry-based metabolomics for irritable bowel syndrome biomarkers. Therap Adv Gastroenterol 12, 1756284819886425 (2019).
20. Baj, A., et al. Glutamatergic Signaling Along The Microbiota-Gut-Brain Axis. Int J Mol Sci 20(2019).

1. A method of treatment or prevention of irritable bowel syndrome (IBS), the method comprising:
administering a preparation comprising at least one probiotic strain Lactobacillus plantarum (Lactiplantibacillus plantarum) DSM 33363.

2. The method of treatment or prevention according to claim 1, wherein the preparation further comprises:
one or more probiotic strains selected from the group consisting of: Lactobacillus plantarum (Lactiplantibacillus plantarum) DSM 33364, Lactobacillus paracasei (Lacticaseibacillus paracasei) DSM 33373, Lactobacillus reuteri (Limosilactobacillus reuteri) DSM 33374, Bacillus megaterium (Priestia megaterium) DSM 33300, Bacillus pumilus DSM 33297, and Bacillus pumilus DSM 33355.

3. The method of treatment or prevention according to claim 1, wherein the IBS is independent from ingestion and/or gastrointestinal metabolization of gluten.

4. The method of treatment or prevention according to claim 1, wherein the at least one probiotic strain is present in a dormant form or as one or more vegetative cell.

5. The method of treatment or prevention according to claim 1, wherein at least one cytoplasmic extractor at least one cell-free supernatant or heat-killed biomass of the at least one probiotic strain is used.

6. The method of treatment or prevention according to claim 1, wherein the preparation further comprises one or more selected from the group consisting of: microbial proteases purified from Aspergillus niger, Aspergillus oryzae, Bacillus sp., Lactobacillus sp., Pediococcus sp., Weissella sp., Rothia mucilaginosa, Rothia aeria, subtilisins, nattokinase, and digestive enzymes.

7. The method of treatment or prevention according to claim 1, wherein the preparation further comprises a substance, which acts as permeabilizer of the microbial cell membrane of members of Bacillus sp., Lactobacillus sp., Pediococcus sp., and Weissella sp.

8. The method of treatment or prevention according to claim 1, wherein the at least one probiotic strain is immobilized individually or as consortia.

9. The method of treatment or prevention according to claim 1, wherein the preparation is at least one selected from the group consisting of: a food, feed supplement, functional food, food product, and pharmaceutical product.

10. The method of treatment or prevention according to claim 1, wherein said preparation is formulated for oral use.

11. A foodstuff composition comprising a preparation comprising at least one probiotic strain Lactobacillus plantarum (Lactiplantibacillus plantarum) DSM 33363- and at least one further ingredient selected from the group consisting of: proteins, carbohydrates, fats, further probiotics, prebiotics, enzymes, vitamins, immune modulators, milk replacers, minerals, amino acids, coccidiostats, acid-based products, and medicines, and combinations thereof.

12. A method of treatment or prevention of abdominal pain and/or disordered defecation comprising:
administering a preparation comprising at least one probiotic strain Lactobacillus plantarum (Lactiplantibacillus plantarum) DSM 33363.

13. A method of treatment or prevention of intolerance towards at least one selected from the group consisting of fermentable oligosaccharides, disaccharides, monosaccharides, and polyols (FODMAP intolerance), the method comprising:
administering a preparation comprising at least one probiotic strain Lactobacillus plantarum (Lactiplantibacillus plantarum) DSM 33363.

14. The method of treatment or prevention according to claim 6 wherein the at least one digestive enzyme is selected from the group consisting of: lactase, glutenase, gliadin hydrolase, protein hydrolase, carbohydrate hydrolase, lipid hydrolase, and lipase.

15. The method of treatment or prevention according to claim 10, wherein the preparation is formulated as at least one selected from the group consisting of: pills, capsules, tablets, granular powders, opercula, soluble granules, bags, drinkable vials, syrup, and a beverage, and/or the preparation is added to food; optionally wherein the food is at least one selected from the group consisting of: cereals, gummies, bread, muesli, muesli bars, health bars, biscuits, chocolates, yoghurts, and spreads.

16. The method of treatment or prevention according to claim 12, the method comprising:
administering the preparation in combination with one or more of the probiotic strains selected from the group consisting of: Lactobacillus plantarum (Lactiplantibacillus plantarum) DSM 33364, Lactobacillus paracasei (Lacticaseibacillus paracasei) DSM 33373, Lactobacillus reuteri (Limosilactobacillus reuteri) DSM 33374, Bacillus megaterium (Priestia megaterium) DSM 33300, Bacillus pumilus DSM 33297, and Bacillus pumilus DSM 33355.

17. The method of treatment or prevention according to claim 13, the method comprising:
administering the preparation in combination with one or more of the probiotic strains selected from the group consisting of: Lactobacillus plantarum (Lactiplantibacillus plantarum) DSM 33364, Lactobacillus paracasei (Lacticaseibacillus paracasei) DSM 33373, Lactobacillus reuteri (Limosilactobacillus reuteri) DSM 33374, Bacillus megaterium (Priestia megaterium) DSM 33300, Bacillus pumilus DSM 33297, and Bacillus pumilus DSM 33355.
